(19) United States
(12) Patent Application Publication
(10) Pub. No.: US 2014/0228640 A1
(43) Pub. Date: Aug. 14, 2014

(54) Laryngeal Mask Assembly

(71) Applicants: Haim Breslauer, Petach Tikva (IL); Shlomo Besharim, Omer (IL)

(72) Inventors: Haim Breslauer, Petach Tikva (IL); Shlomo Besharim, Omer (IL)

(73) Assignee: Breslauer Ltd., Petach Tikva (IL)

(21) Appl. No.: 14/128,172

(22) PCT Filed: Jun. 28, 2012
(86) PCT No.: PCT/IL12/00267

§ 371 Date: Apr. 1, 2014

(30) Foreign Application Priority Data
Jun. 28, 2011 (GB) 1111021.0

(51) Int. Cl. A61M 16/04; A61B 1/267; A61B 1/00

(52) U.S. Cl. CPC A61M 16/0427; A61M 16/0447; A61B 1/00154; A61B 1/2673
USPC 600/115

(57) ABSTRACT

An artificial airway assembly for ventilating a patient, is provided, comprising a flexible airway tube having a proximal end and a distal end, adapted to guide a working tool to an operating position to view vocal cords and trachea of the patient, said flexible airway tube comprising at least one locally weakened and tearable course adapted to permit detachment of at least a section of said airway tube while maintaining said working tool in said operating position; and a mask fixedly secured to said distal end of said flexible airway tube, said mask having conforming means for conforming to a space immediately adjacent to the larynx of the patient to properly position said working tool. In alternative embodiments, the assembly further includes an inflatable balloon that directs the airway tube toward the patient's trachea.

### FIELD OF THE INVENTION

This invention relates to artificial airway assemblies. In particular, it relates to artificial airway assemblies in which the airway tube is designed for ease of placement and removal.

### BACKGROUND OF THE INVENTION

U.S. Pat. No. 4,509,514 discloses an artificial airway device that comprises a laryngeal mask (LMA). This device is used to facilitate lung ventilation in unconscious patients. In contrast to devices that were known in the art at the time, the device disclosed in U.S. Pat. No. 4,509,514 was designed to prevent airway obstruction, to permit either controlled or spontaneous inhalation, and to prevent inhalation into the lungs of extraneous matter. In addition, it was designed to seal around the circumference of the laryngeal inlet without penetrating (and possibly damaging) the larynx.

Since that time, numerous advances have been made in the design of artificial airway technology. For example, U.S. Pat. No. RE35,531 comprises a mask that is capable of conforming to the space immediately adjacent to the larynx in order to properly position the working tool that passes through the flexible airway tube, and in which the artificial airway assembly is characterized by removing means for removing the flexible airway tube from the working tool while maintaining the tool in its operating position.

U.S. Pat. No. 6,761,170 discloses a laryngeal mask assembly that includes a blocker that is operable to prevent entry of the epiglottis into the cavity during insertion of the assembly but that does not hinder gas passage along the assembly after insertion. In some embodiments of the invention, this blocker is an inflatable balloon.

While these inventions include passive blocking means for preventing the device or the working tool from entering the esophagus, these means are not guaranteed to prevent accidental insertion of the working tool or its guide tube into the esophagus. A laryngeal mask assembly that comprises active means for manipulating the tubing into the trachea still remains a long-standing but unmet need.

Furthermore, it is still a long-standing but unmet need to provide a laryngoscope in which the airway tube can be easily detached after the working tool has been inserted. Such detachment would be advantageous if the laryngoscope could be separated into relatively small sections. In other words, it would be advantageous if there would be numerous tearing courses which run along the longitudinal axis of said laryngoscope, such that the dimensions of the separated sections of said laryngoscope are minimized.

### SUMMARY OF THE INVENTION

The laryngeal mask assembly herein disclosed is designed to meet this need. It is therefore an object of the present invention to disclose an artificial airway assembly for ventilating a patient, the artificial airway assembly comprising a flexible airway tube having a proximal end and a distal end, the proximal end comprising a pass-through means for allowing a working tool to pass there through and for guiding the working tool to an operating position to view vocal cords and trachea of the patient, the flexible airway tube comprising at least one locally weakened and tearable course adapted to permit detachment of at least a section of the airway tube while maintaining the working tool in the operating position; and a mask fixedly secured securely fixed to the distal end of the flexible airway tube, the mask having conforming means for conforming to a space immediately adjacent to the larynx of the patient to properly position the working tool.

It is one object of the present invention to provide an artificial airway assembly for ventilating a patient, the artificial airway assembly comprising: a flexible airway tube having a proximal end and a distal end interconnected by a main longitudinal axis, adapted to guide a working tool to an operating position; and, a mask coupled to the distal end of the flexible airway tube; wherein the flexible airway tube comprises at least two courses having predetermined shape, such that at least two separate sections of the airway tube are detachable from the same while maintaining the working tool in the operating position.

It is another object of the present invention to provide the artificial airway assembly as defined above, wherein the at least two courses are complementary to and conform to each other.

It is another object of the present invention to provide the artificial airway assembly as defined above, wherein the courses are locally weakened and tearable courses.

It is another object of the present invention to provide the artificial airway assembly as defined above, wherein the courses pass distally along the flexible airway, such that the dimensions of the at least two sections detached from the airway tube are minimized.

It is another object of the present invention to provide the artificial airway assembly as defined above, wherein at least one of the courses is disposed along the main longitudinal axis.

It is another object of the present invention to provide the artificial airway assembly as defined above, wherein the at least two courses are disposed independently about the circumference of the flexible airway.

It is another object of the present invention to provide the artificial airway assembly as defined above, wherein at least one of the courses is disposed spirally about the circumference of the flexible airway.

It is another object of the present invention to provide the artificial airway assembly as defined above, wherein the courses are homogeneously disposed along the circumference of the flexible airway.

It is another object of the present invention to provide the artificial airway assembly as defined above, wherein the courses are characterized by having two edges overlapping each other.

It is another object of the present invention to provide the artificial airway assembly as defined above, wherein the courses are heterogeneously disposed along the circumference of the flexible airway.

It is another object of the present invention to provide the artificial airway assembly as defined above, wherein the distance between each pair of the plurality of courses is equal.

It is another object of the present invention to provide the artificial airway assembly as defined above for various LMAs, such as LMA No. 1 (internal diameter of about 4 mm) to LMA No. 5 (internal diameter of about 15 mm). The distance (along a spread tube) between each pair of the plurality of courses is greater than about 4 mm and smaller than about 50 mm. The term ‘about’ refers to a measure being ±50% of the defined value.

It is another object of the present invention to provide the artificial airway assembly as defined above, wherein the distance between each pair of the courses is optimized, such that the dimensions of the sections detached from the airway tube are minimized.

It is another object of the present invention to provide the artificial airway assembly as defined above, wherein the distance is calculated according to the artificial airway assembly minimization function F_{Min}(x); the F_{Min}(x) is a function of the number of the courses. n; the circumference of the flexible airway tube, C; volume of the flexible airway tube, V; the distance between each pair of the courses. D, or any combination thereof; represented by F_{Min}(n, C, V, D).

It is another object of the present invention to provide the artificial airway assembly as defined above, wherein the mask comprises at least two courses having predetermined shape, such that at least two separate sections of the airway tube are detachable from the same while maintaining the working tool in the operating position.

It is another object of the present invention to provide the artificial airway assembly as defined above, wherein the at least two courses are complementary to and conform to each other.

It is another object of the present invention to provide the artificial airway assembly as defined above, wherein the courses are locally weakened and tearable courses.

It is another object of the present invention to provide the artificial airway assembly as defined above, wherein the courses are provided so as to minimize the dimensions of the at least one section detached from the mask.

It is another object of the present invention to provide the artificial airway assembly as defined above, wherein the at least one of the courses is disposed independently about the circumference of the mask.

It is another object of the present invention to provide the artificial airway assembly as defined above, wherein the courses are disposed spirally about the circumference of the mask.

It is another object of the present invention to provide the artificial airway assembly as defined above, wherein the courses are characterized by having two edges overlapping each other.

It is another object of the present invention to provide the artificial airway assembly as defined above, wherein the mask is fixedly secured to the operating position of the flexible airway tube.

It is another object of the present invention to provide the artificial airway assembly as defined above, wherein the flexible airway tube is branched.

It is another object of the present invention to provide the artificial airway assembly as defined above, wherein the flexible airway tube is adapted for guiding the working tool to an operating position to view vocal cords and trachea of the patient.

It is another object of the present invention to provide the artificial airway assembly as defined above, further comprising at least one inflatable balloon disposed about, and in physical contact with, one side of the flexible airway, whereby upon inflation of the inflatable balloon while the artificial airway assembly is in place, at least part of the flexible airway missing verb in a predetermined direction.

It is another object of the present invention to provide the artificial airway assembly as defined above, wherein the predetermined direction is in the direction of the trachea of the patient.

It is another object of the present invention to provide the artificial airway assembly as defined above, wherein the inflatable balloon is disposed externally to the flexible airway.

It is another object of the present invention to provide the artificial airway assembly as defined above, wherein the inflatable balloon is disposed within the flexible airway.

It is another object of the present invention to provide the artificial airway assembly as defined above, wherein the inflatable balloon is integrated within the flexible airway.

It is another object of the present invention to provide the artificial airway assembly as defined above, wherein the mask is fixedly secured to the distal end of the flexible airway tube, the mask having conforming means for conforming to a space immediately adjacent to the larynx of the patient to properly position the working tool.

It is another object of the present invention to provide the artificial airway assembly as defined above, wherein the predetermined shape of the courses is selected from a group consisting of Zig-Zag shape, waved, sinus, triangular waved shaped, square waved shaped, continuous, discontinuous or any combination thereof.

It is another object of the present invention to provide the artificial airway assembly as defined above, additionally comprising a maneuvering mechanism adapted to reorient the distal end of the flexible airway tube.

It is another object of the present invention to provide the artificial airway assembly as defined above, comprising a flexible airway tube having a proximal end and a distal end, the proximal end comprising a pass-through means for allowing a working tool to pass there through and for guiding the working tool to an operating position to view vocal cords and trachea of the patient, the flexible airway tube comprising at least two locally weakened and tearable courses adapted to permit detachment of at least a section of the airway tube while maintaining the working tool in the operating position; at least one airway-directing mechanism disposed on one side of and in physical contact with the flexible airway tube; and a mask coupled to the distal end of the flexible airway tube; wherein activation of the airway-directing mechanism while the artificial airway assembly is in place, is directing at least part of the flexible airway to a predetermined location.

It is another object of the present invention to provide the artificial airway assembly as defined above, wherein the airway-directing mechanism is selected from a group consisting of pneumatic, mechanic and pneumatic-mechanic integrated mechanism.

It is another object of the present invention to provide the artificial airway assembly as defined above, wherein the pneumatic airway-directing mechanism comprising at least one inflatable balloon useful to direct at least part of the flexible airway to a predetermined location.

It is another object of the present invention to provide the artificial airway assembly as defined above, wherein the mechanic airway-directing mechanism comprising at least one maneuverable effecter useful to direct at least part of the flexible airway to a predetermined location.

It is another object of the present invention to provide an artificial airway assembly for ventilating a patient, the artificial airway assembly (10) comprising: a flexible airway tube having a proximal end and a distal end interconnected by a main longitudinal axis, adapted to guide a working tool to an operating position; at least one inflatable balloon disposed on one side of and in physical contact with the flexible airway tube; and, a mask coupled to the distal end of the flexible airway tube; wherein inflation of the inflatable balloon while the artificial airway assembly is in place, is adapted to direct at least part of the flexible airway to a predetermined location.

It is another object of the present invention to provide the artificial airway assembly as defined above, wherein the predetermined location is the trachea of the patient.

It is another object of the present invention to provide the artificial airway assembly as defined above, wherein the inflatable balloon is disposed externally to the flexible airway.

It is another object of the present invention to provide the artificial airway assembly as defined above, wherein the inflatable balloon is disposed within the flexible airway.

It is another object of the present invention to provide the artificial airway assembly as defined above, wherein the flexible airway tube comprises at least two courses having predetermined shape, such that at least two separate sections of the airway tube are detachable from the same while maintaining the working tool in the operating position.

It is another object of the present invention to provide the artificial airway assembly as defined above, wherein the at least two courses are complementary to and conform to each other.

It is another object of the present invention to provide the artificial airway assembly as defined above, wherein the courses are locally weakened and tearable courses.

It is another object of the present invention to provide the artificial airway assembly as defined above, wherein the courses pass distally along the flexible airway, such that the dimensions of the at least two sections detached from the airway tube are minimized.

It is another object of the present invention to provide the artificial airway assembly as defined above, wherein at least one of the courses is disposed along the main longitudinal axis.

It is another object of the present invention to provide the artificial airway assembly as defined above, wherein the at least two courses are disposed independently about the circumference of the flexible airway.

It is another object of the present invention to provide the artificial airway assembly as defined above, wherein the at least one of the courses is disposed spirally about the circumference of the flexible airway.

It is another object of the present invention to provide the artificial airway assembly as defined above, wherein the plurality of courses is homogeneously disposed along the circumference of the flexible airway.

It is another object of the present invention to provide the artificial airway assembly as defined above, wherein the plurality of courses is heterogeneously disposed along the circumference of the flexible airway.

It is another object of the present invention to provide the artificial airway assembly as defined above, wherein the courses are characterized by having two edges overlapping each other.

It is another object of the present invention to provide the artificial airway assembly as defined above, wherein the distance between each pair of the plurality of courses is equal.

It is another object of the present invention to provide the artificial airway assembly as defined above, wherein the distance between each pair of the courses is optimized, such that the dimensions of the sections detached from the airway tube are minimized.

It is another object of the present invention to provide the artificial airway assembly as defined above, wherein the distance is calculated according to the artificial airway assembly minimization function F_{Min}(x); the F_{Min}(x) is a function of the number of the courses. n; the circumference of the flexible airway tube, C; volume of the flexible airway tube, V; the distance between each pair of the courses. D, or any combination thereof; represented by F_{Min}(n, C, V, D).

It is another object of the present invention to provide the artificial airway assembly as defined above, wherein the mask comprises at least two courses having predetermined shape, such that at least two separate sections of the airway tube are detachable from the same while maintaining the working tool in the operating position.

It is another object of the present invention to provide the artificial airway assembly as defined above, wherein the at least two courses are complementary to and conform to each other.

It is another object of the present invention to provide the artificial airway assembly as defined above, wherein the courses are locally weakened and tearable courses.

It is another object of the present invention to provide the artificial airway assembly as defined above, wherein the courses are provided so as to minimize the dimensions of the at least one section detached from the mask.

It is another object of the present invention to provide the artificial airway assembly as defined above, wherein the at least one of the courses is disposed independently about the circumference of the mask.

It is another object of the present invention to provide the artificial airway assembly as defined above, wherein the at least one of the courses is disposed spirally about the circumference of the flexible airway.

It is another object of the present invention to provide the artificial airway assembly as defined above, wherein the courses are characterized by having two edges overlapping each other.

It is another object of the present invention to provide the artificial airway assembly as defined above, wherein the mask is fixedly secured to the operating position of the flexible airway tube.

It is another object of the present invention to provide the artificial airway assembly as defined above, wherein the flexible airway tube is branched.

It is another object of the present invention to provide the artificial airway assembly as defined above, wherein the flexible airway tube is adapted for guiding the working tool to an operating position to view vocal cords and trachea of the patient.

It is another object of the present invention to provide the artificial airway assembly as defined above, wherein mask coupled fixedly secured to the distal end of the flexible airway tube, the mask having conforming means for conforming to a space immediately adjacent to the larynx of the patient to properly position the working tool.

It is another object of the present invention to provide the artificial airway assembly as defined above, wherein the predetermined shape of the courses is selected from a group consisting of Zig-Zag shape, waved, sinus, triangular waved shaped, square waved shaped, continuous, discontinuous or any combination thereof.

It is another object of the present invention to provide the artificial airway assembly as defined above, additionally comprising a maneuvering mechanism adapted to reorient the distal end of the flexible airway tube.

It is another object of the present invention to provide an artificial airway assembly for ventilating a patient, the artificial airway assembly comprising: a flexible airway tube having a proximal end and a distal end, the proximal end comprising a pass-through means for allowing a working tool to pass there through and for guiding the working tool to an operating position to view vocal cords and trachea of the patient;

at least one airway-directing mechanism disposed on one side of and in physical contact with the flexible airway tube; and, a mask coupled to the distal end of the flexible airway tube; wherein the flexible airway tube comprises a dorsal section and a ventral section; the flexible airway tube has a drawer-like configuration for detachment of at least a section of the airway tube while maintaining the working tool in the operating position; further wherein the activation of the airway-directing mechanism while the artificial airway assembly is in place, is directing at least part of the flexible airway to a predetermined location.

It is another object of the present invention to provide the artificial airway assembly as defined above, wherein the drawer like configuration of the flexible airway tube comprises a drawn section allowing extraction of the mask tube while maintaining the flexible airway tube in the operating position.

It is another object of the present invention to provide the artificial airway assembly as defined above, wherein the drawer like configuration of the flexible airway tube is characterized by having two connecting sections overlapping each other.

It is another object of the present invention to provide the artificial airway assembly as defined above, wherein the detachment section of the airway tube is the ventral section.

It is another object of the present invention to provide the artificial airway assembly as defined above, wherein the detachment of at least a section of the airway tube is preformed in manner selected from the group consisting of: a sliding mechanism, a lego-like mechanism, a pull out mechanism, a quick release mechanism, or any disengaging mechanism.

It is another object of the present invention to provide a method for intubating a patient using an artificial airway assembly, the method comprising a step of a. obtaining a flexible airway tube having a proximal end and a distal end interconnected by a main longitudinal axis; adapted to guide a working tool to an operating position; the flexible airway tube comprising at least two courses. having predetermined shape, along which at least two sections of the airway tube are detached while maintaining the working tool in the operating position; b. fixedly securing a mask to the distal end of the flexible airway tube; c. inserting the artificial airway assembly into the mouth of a patient; d. positioning the mask adjacent to the larynx; e. moving the working tool through the flexible airway tube at least to the distal end of the artificial airway assembly and the mask; f. at least partially tearing the flexible airway tube along the at least two courses. Thereby detaching at least two sections of the airway tube while maintaining the working tool in the operating position; and, g. removing the artificial airway assembly from the mouth of the patient.

It is another object of the present invention to provide the method as defined above, additionally comprising the step of initiating ventilation of the patient via the proximal end.

It is another object of the present invention to provide the method as defined above, in which the step of moving the working tool through the flexible airway tube further comprises a step of moving the working tool beyond the mask and into the trachea of the patient.

It is another object of the present invention to provide the method as defined above, wherein the step of removing the artificial airway assembly from the working tool further comprises steps of: tearing the flexible airway tube along each of the courses and, removing the artificial airway assembly in a piecewise fashion as each piece is detached.

It is another object of the present invention to provide the method as defined above, wherein the at least two courses are complementary to and conform to each other.

It is another object of the present invention to provide the method as defined above, wherein the courses are locally weakened and tearable courses.

It is another object of the present invention to provide the method as defined above, wherein the artificial airway assembly further comprises an inflatable balloon disposed about one side of the flexible airway tube, the method further comprising a step of at least partially inflating the balloon whereby the flexible airway tube is directed toward the trachea of the patient.

It is another object of the present invention to provide the method as defined above, further including a step of deflating the inflatable balloon.

It is another object of the present invention to provide the method as defined above, additionally comprising a step of disposing the courses along the flexible airway, such that the dimensions of the two sections detached from the airway tube are minimized.

It is another object of the present invention to provide the method as defined above, additionally comprising a step of disposing at least one of the courses along the main longitudinal axis.

It is another object of the present invention to provide the method as defined above, additionally comprising a step of disposing the courses independently about the circumference of the flexible airway.

It is another object of the present invention to provide the method as defined above, additionally comprising a step of disposing the courses comprises are disposed spirally about the circumference of the flexible airway.

It is another object of the present invention to provide the method as defined above, additionally comprising a step of homogeneously disposing the courses along the circumference of the flexible airway.

It is another object of the present invention to provide the method as defined above, additionally comprising a step of heterogeneously disposing the plurality of courses along the circumference of the flexible airway.

It is another object of the present invention to provide the method as defined above, additionally comprising a step of providing the courses by having two edges overlapping each other.

It is another object of the present invention to provide the method as defined above, wherein the distance between each pair of the courses is equal.

It is another object of the present invention to provide the method as defined above, wherein the distance between each pair of the courses is optimized, such that the dimensions of the sections detached from the airway tube are minimized.

It is another object of the present invention to provide the method as defined above, wherein the distance is calculated according to the artificial airway assembly minimization function F_{Min}(x); the F_{Min}(x) is a function of the number of the courses. n; the circumference of the flexible airway tube, C; volume of the flexible airway tube, V; the distance between each pair of the courses. D, or any combination thereof; represented by F_{Min}(n, C, V, D).

It is another object of the present invention to provide the method as defined above, additionally comprising a step of providing the mask with at least two courses having predetermined shape, such that at least two separate sections of the airway tube are detachable from the same while maintaining the working tool in the operating position.

It is another object of the present invention to provide the method as defined above, wherein the at least two courses are complementary to and conform to each other.

It is another object of the present invention to provide the method as defined above, wherein the courses are locally weakened and tearable courses.

It is another object of the present invention to provide the method as defined above, additionally comprising a step of providing the courses, so as to minimize the dimensions of the at least one section detached from the mask.

It is another object of the present invention to provide the method as defined above, wherein at least one of the courses are disposed independently about the circumference of the mask.

It is another object of the present invention to provide the method as defined above, additionally comprising a step of securing the mask to the operating position of the flexible airway tube.

It is another object of the present invention to provide the method as defined above, wherein the flexible airway tube is branched.

It is another object of the present invention to provide the method as defined above, wherein the flexible airway tube is adapted for guiding the working tool to an operating position to view vocal cords and trachea of the patient.

It is another object of the present invention to provide the method as defined above, additionally comprising a step of providing at least one inflatable balloon disposed about, and in physical contact with, one side of the flexible airway.

It is another object of the present invention to provide the method as defined above, additionally comprising a step of, upon inflation of the inflatable balloon while the artificial airway assembly is in place, directing at least part of the flexible airway in the direction of the trachea of the patient.

It is another object of the present invention to provide the method as defined above, additionally comprising a step of disposing the inflatable balloon externally to the flexible airway.

It is another object of the present invention to provide the method as defined above, additionally comprising a step of disposing the inflatable balloon within the flexible airway.

It is another object of the present invention to provide the method as defined above, additionally comprising a step of integrating the inflatable balloon within the flexible airway.

It is still object of the present invention to provide the method as defined above, additionally comprising a step of providing the mask with conforming means for conforming to a space immediately adjacent to the larynx of the patient to properly position the working tool.

It is lastly an object of the present invention to provide the method as defined above, wherein the predetermined shape of the courses is selected from a group consisting of Zig-Zag shape, waved, sinus, triangular waved shaped, square waved shaped, continuous, discontinuous or any combination thereof.

It is lastly an object of the present invention to provide the method as defined above, additionally comprising step of maneuvering and reorienting the distal end of the flexible airway tube.

### BRIEF DESCRIPTION OF THE DRAWINGS

The invention is now described with reference to the drawings, wherein

FIG. 1 is a cross-sectional view of a laryngeal mask assembly according to one embodiment of the invention;

FIG. 2 is a cross-sectional view of the flexible airway tube of a laryngeal mask assembly in place according to a second embodiment of the invention;

FIG. 3 is a side view, partially cut away, of a laryngeal mask assembly in its operating position according to the embodiment illustrated in FIG. 1;

FIG. 4 is a side view, partially cut away, of a laryngeal mask assembly in its operating position according to the embodiment of the invention illustrated in FIG. 2;

FIG. 5 is a side view, partially cut away, of a laryngeal mask assembly in its operating position according to a third embodiment of the invention;

FIGS. 6-7 illustrate different shapes of the courses 301.

FIGS. 8-10 illustrate another embodiment of the present invention which illustrate a maneuvering mechanism (upwards and downwards) to the end of the tubus; and,

FIGS. 11-15 illustrate another embodiment of the present invention which illustrate the drawer-like configuration of the mask tube.

### DETAILED DESCRIPTION OF THE PREFERRED EMBODIMENTS

In the following description, various aspects of the invention will be described. For the purposes of explanation, specific details are set forth in order to provide a thorough understanding of the invention. It will be apparent to one skilled in the art that there are other embodiments of the invention that differ in details without affecting the essential nature thereof. Therefore the invention is not limited by that which is illustrated in the figures and described in the specification, but only as indicated in the accompanying claims, with the proper scope determined only by the broadest interpretation of the claims.

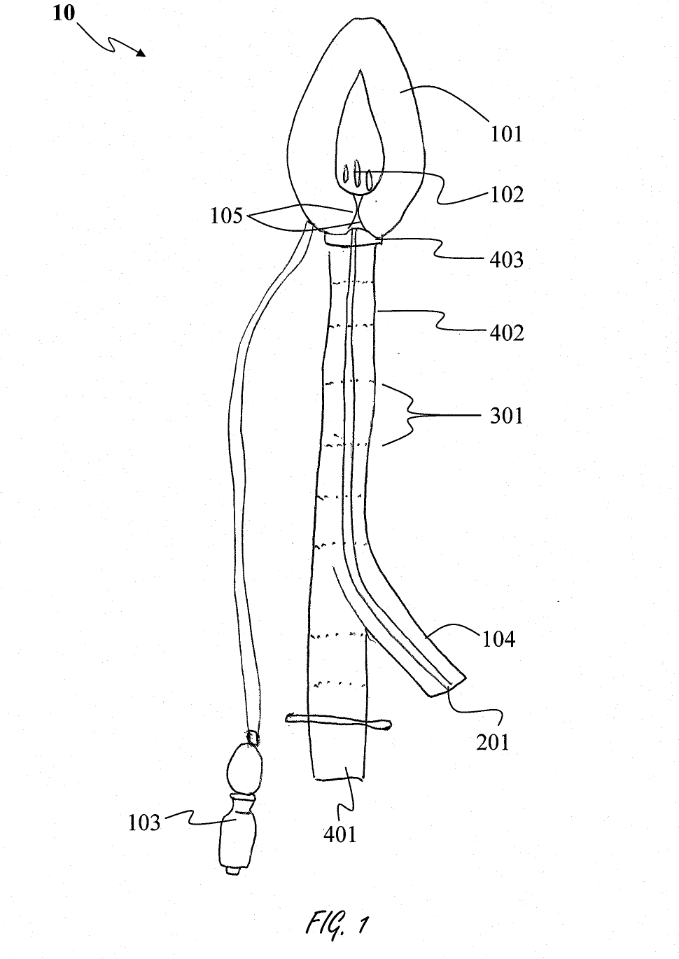

Reference is now made to FIG. 1, which shows a cross-sectional view of an artificial airway assembly according to one embodiment 10 of the present invention. Artificial airway assembly 10 comprises a flexible airway tube (i.e., a tubus) 402, which comprises a proximal end 401 and a distal end 403. In preferred embodiments, the flexible airway tube comprises a second branch 104 with a channel 201 passing there through. The second branch guides a working tool 202 (not shown in FIG. 1) into the flexible airway tube and allows for ventilation to continue without interruption through the flexible airway tube. The working tool may be any surgical tool or device adapted for introduction into a patient's trachea (e.g. a fiber optic cable for determining the proper location of distal end 403).

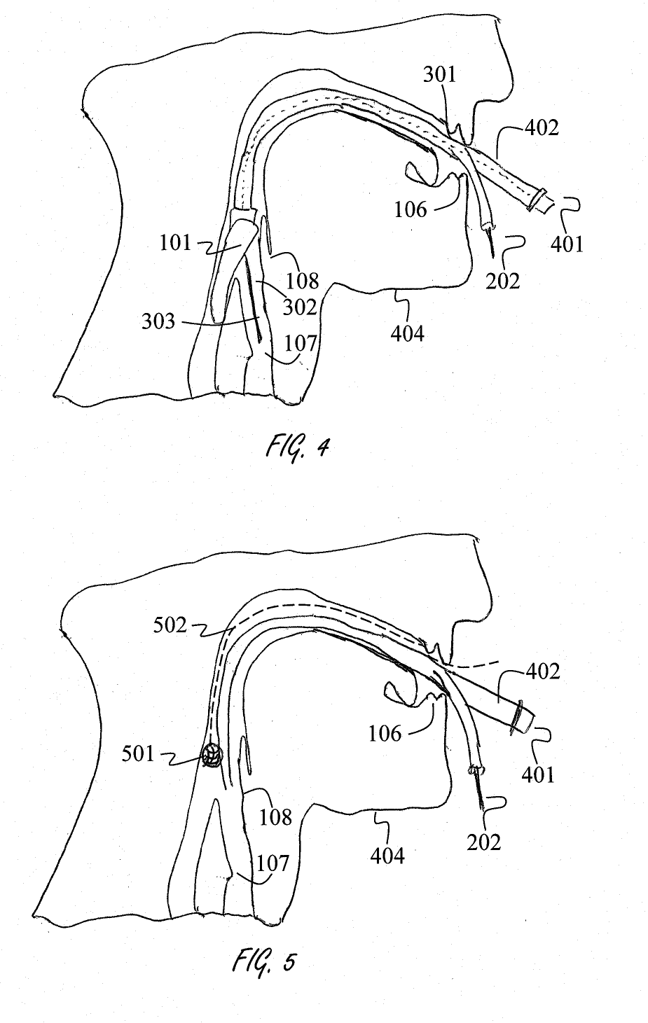

A mask 101 is fixedly secured to the distal end of the flexible airway tube. Mask 101 incorporates at least one hole 102 for allowing air and the working tool to pass there through. The mask is adapted to conform to the space immediately adjacent to the larynx of the patient. In preferred embodiments, the mask is constructed of a resilient flexible biocompatible material in the form of a peripheral ring capable of defining a hollow space, capable of being inflated and deflated to accommodate for the space adjacent to the larynx. When installed, the mask adopts a configuration substantially conforming to the shape of the laryngeal inlet (cf. FIGS. 3-5). A valve assembly 103 is in fluid connection with the mask and allows air to pass in and out of the mask, allowing for easier insertion, as the mask need not be inflated until it is already substantially in place. In typical embodiments, air is injected into or withdrawn from mask 101 by use of a syringe, but any means known in the art may be used instead. The inflatable portion of the mask has ends 105 that abut one another upon inflation, thus providing a peripheral seal. When the mask is in its deflated state, the two ends separate to facilitate removal of the device and the working tool.

The flexible airway tube is further characterized by at least one locally weakened and tearable course 301 adapted to permit detachment of at least a section of the airway tube while maintaining the working tool in the operating position. The weakened and tearable courses may be of any shape and configuration suitable for detaching the airway tube from the working tool. For example, they may consist of slits cut partway through the wall of the airway tube, or perforations in the tube. In the embodiment shown in FIG. 1, the weakened and tearable courses are disposed about the circumference of the airway tube in a plurality of locations along its length. In this embodiment, pulling on the airway tube with sufficient strength will cause the proximal weakened and tearable course to tear, thus detaching the proximal portion of the airway tube from the remainder, enabling piecewise removal of the airway tube while leaving the working tool in place.

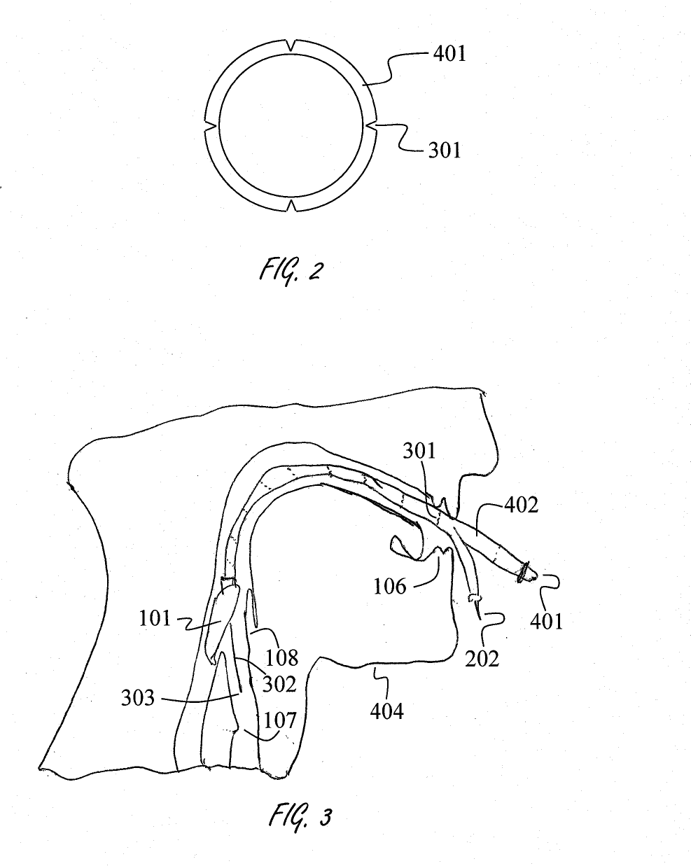

Reference is now made to FIG. 2, which shows a cross-sectional view of the airway tube of a second embodiment of the invention. In this embodiment, the weakened and tearable courses are disposed longitudinally along the airway tube at several locations along its circumference. In these embodiments, the airway tube can be removed by application of force perpendicular to the longitudinal axis of the airway tube, thus separating it into sections that can be removed individually while leaving the working tool in place.

In a third embodiment, not illustrated, the weakened and tearable course is disposed as a single spiral down the length and around the circumference of the airway tube. A pull in the direction of the longitudinal axis will separate the airway tube along this spiral, making it possible to remove the entire airway tube in a single piece, while leaving the working tool in its operating position.

The three embodiments of the weakened and tearable course described in detail above are presented for exemplary purposes only in order to illustrate to one skilled in the art the principles of construction of the invention, and not to limit the invention to those specific embodiments. It is contemplated by the inventor that all equivalent means for providing weakened and tearable courses that enable easy detachment of at least part of the flexible airway tube are within the scope of the invention.

Reference is now made to FIG. 3, which shows a partially cut away view of a preferred embodiment of the invention shown in FIG. 1 in its operating position as it is used to intubate patient 404. The flexible airway tube is inserted into mouth 106 of the patient. Mask 101 is positioned adjacent to the larynx 108, thus sealing at least partially the airway tube for communication exclusively through the laryngeal inlet. Working tool 202 is shown after insertion through the second branch of the flexible airway tube through mask 101 and into the trachea 303 in a position from which a viewing device (e.g. a fiber optic device) can be used to observe the vocal chords 107. In the figure, distal end 302 of the working tool is shown in position above the vocal chords.

Subsequent removal of the airway tube is accomplished by application of force sufficient to detach the proximal portion of the flexible airway tube, after which it can be removed from the patient. This process is repeated until the entire airway tube has been removed, leaving the working tool in its operating position.

Reference is now made to FIG. 4, which shows a partially cut away view of the embodiment of the invention shown in FIG. 2 in its operating position as it is used to intubate patient 404. The operation of the artificial airway assembly shown in FIG. 4 is essentially identical to that shown in FIG. 3. In this case, however, the removal of the airway tube is accomplished by application of force substantially perpendicular to the longitudinal axis of the airway tube. Application of force in this direction will cause the airway tube to peel away from the working tool and to separate into strips, each of which can then be removed individually.

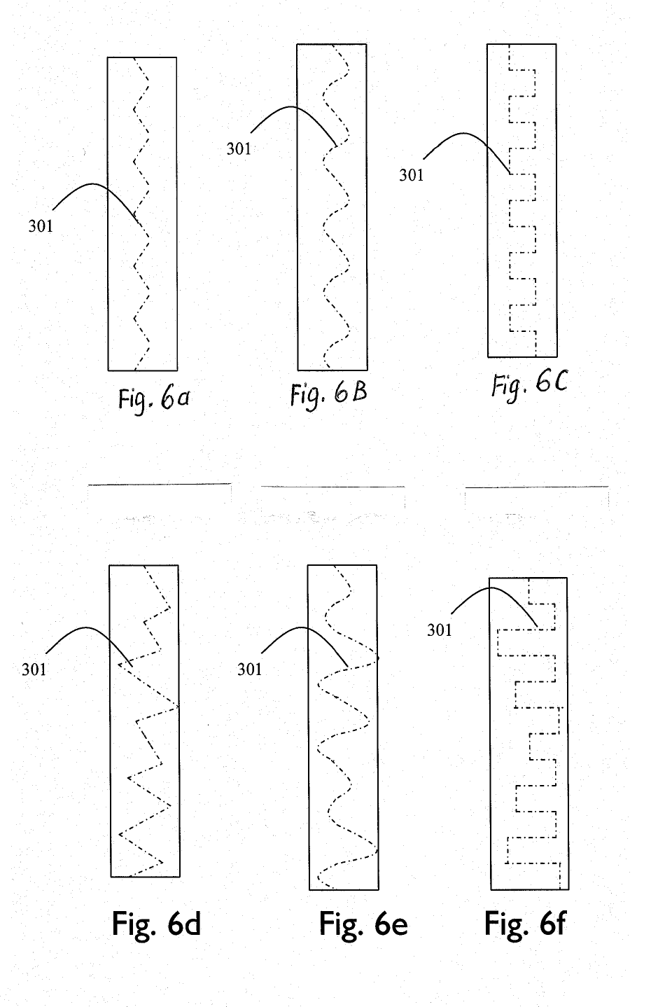

Reference is now made to FIG. 5, which illustrates a third embodiment of the invention in its operating position as is it used to intubate patient 404. For clarity, mask 101 is not shown in the figure. The embodiment of the invention illustrated in FIG. 5 further comprises an inflatable balloon 501 disposed about one side of, and in physical contact with, the flexible airway tube. When the assembly is inserted, inflatable balloon 501 is in its deflated state. In the preferred embodiment of the invention shown in FIG. 5, the assembly further comprises inflation tube 502 in fluid communication with balloon 501. Any other means for inflating the balloon known in the art may be used. After at least partial insertion, the balloon is inflated sufficient to direct the distal end of working tool 202 towards trachea 303, without blocking the flow of air completely. The likelihood of the working tool's accidentally entering the esophagus is thus significantly lowered relative to the likelihood of such an event occurring in artificial airway assemblies known in the art. Balloon 501 may be made of any appropriate biocompatible rubber-like material. In preferred embodiments of the invention, it is disposed on the outer circumference of airway tube 402. In other embodiments, it is disposed within the airway tube.

The balloon and inflation means may be used in conjunction with any of the embodiments of the tearable airway tube disclosed above.

Thus, it is one object of the present invention to provide an artificial airway assembly for ventilating a patient, the artificial airway assembly comprising: a flexible airway tube having a proximal end and a distal end interconnected by a main longitudinal axis, adapted to guide a working tool to an operating position; and, a mask coupled to the distal end of the flexible airway tube, wherein the flexible airway tube comprising at least two courses having predetermined shape, such that at least two separate sections of the airway tube are detachable from the same while maintaining the working tool in the operating position.

Reference is now made to FIGS. 6a-6f illustrating different shape of the courses 301. FIG. 6a illustrates a Zig-Zag shape, waved (FIGS. 6b, & 6e), sinus, triangular waved shaped (FIGS. 6a & 6d), square waved shaped (FIGS. 6c & 6f), continuous or any combination thereof.

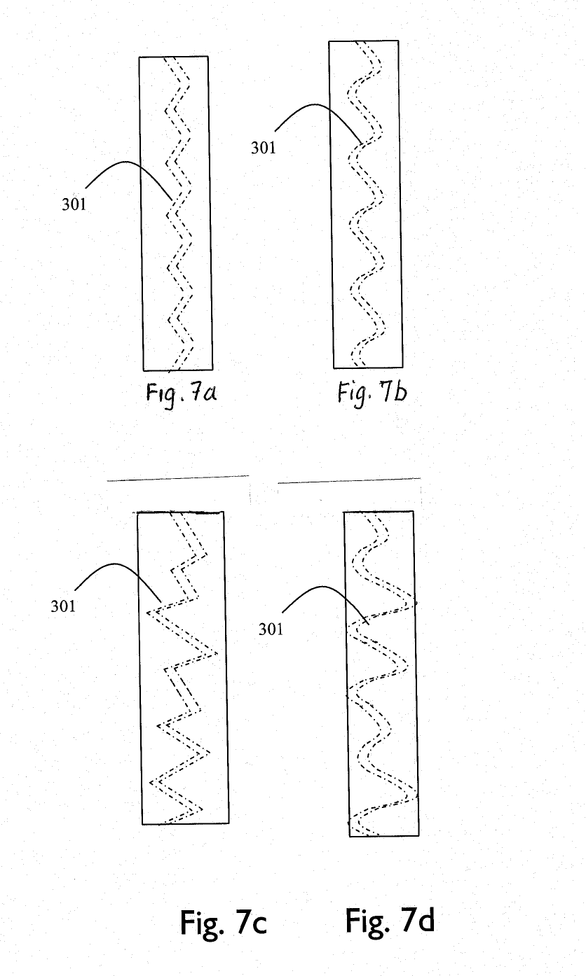

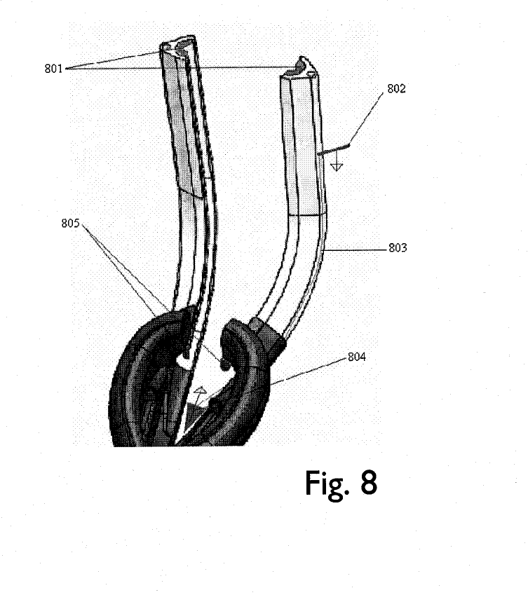

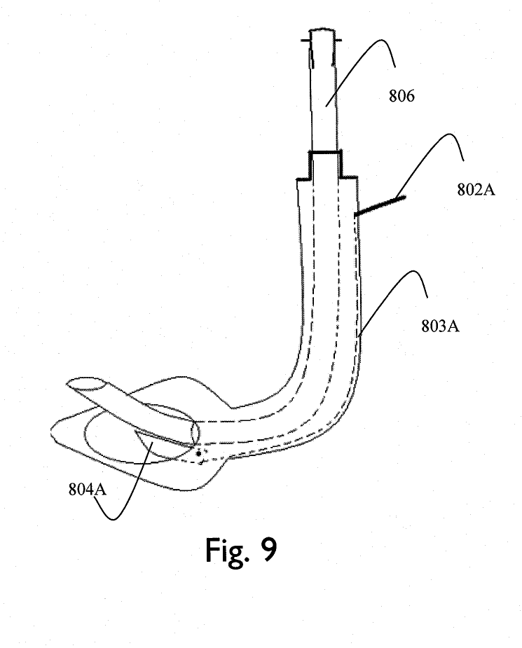

It is another object of the present invention to provide the artificial airway assembly as defined above, wherein the at least two courses are complementary to and conform to each other (see FIGS. 7a-7d).

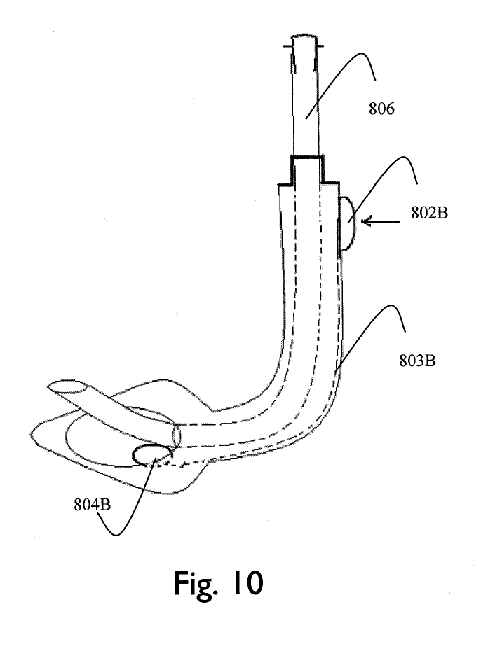

Reference is made to FIGS. 8-10 which illustrate another embodiment of the present invention which illustrate a maneuvering mechanism (upwards and downwards) to the end of the tubus. Numerical reference 801 refers to a silicon ring which enables respiration through the airway tube (tubus) without any leak of gas.

FIGS. 8-10 schematically present an artificial airway assembly. The device comprises, inter alia, a flexible airway tube having a proximal end and a distal end, the proximal end comprising a pass-through means for allowing a working tool to pass there through and for guiding the working tool to an operating position to view vocal cords and trachea of the patient, the flexible airway tube comprising at least two locally weakened and tearable courses adapted to permit detachment of at least a section of the airway tube while maintaining the working tool in the operating position; at least one airway-directing mechanism disposed on one side of and in physical contact with the flexible airway tube; and a mask coupled to the distal end of the flexible airway tube; wherein activation of the airway-directing mechanism while the artificial airway assembly is in place, is directing at least part of the flexible airway to a predetermined location.

The artificial airway assembly defined above, is possibly provided with a pneumatic airway-directing mechanism which comprises at least one inflatable balloon useful to direct at least a part of the flexible airway to a predetermined location. Additionally or alternatively, the artificial airway assembly defined above, is possibly provided with a mechanical airway-directing mechanism which comprises at least one maneuverable effecter useful to direct at least part of the flexible airway to a predetermined location. An artificial airway assembly which comprises an integrated mechanism, namely a pneumatic and mechanical mechanism is possible.

Numerical references 802A and 802B refer thus to a mechanical and pneumatically activation mechanisms: according to a first (mechanical) embodiment of the invention, yet in a non limiting manner, push button coupled to wire 803A, eventually coupled to tongue-like structure (namely, a lingula) 804A. The lingula 804A is adapted to push the tubus end upwards when the pushing button 802A is pressed; according a second (pneumatic) embodiment of the invention, still in a non limiting manner, fluid bag 802B (filled with e.g., water or air) coupled to fluid communication pipe 803B, eventually coupled to tongue-like ligament (namely, a lingula) 804B. The lingula 804B is adapted to push the tubus end upwards when the fluid bag, such as inflatable balloon 802b is pressed and the fluid is compressed. Numerical reference 806 is a tracheal airway tube.

Numerical reference 805 refers to a silicon cushion adapted to press the end of the tubus downwardly.

When pushing button 802 is not pressed the silicon cushion 805 pushed the end of the tubus downwardly. When the pushing button 802 is pressed the lingula 804 overcomes the pressure applied by the silicon cushion 805 and pushes the end of the tubus upwardly. In such a manner the end of the tubus is maneuvered upwards and downwards

It is another object of the present invention to provide the artificial airway assembly as defined above, wherein the courses are locally weakened and tearable courses.

It is another object of the present invention to provide the artificial airway assembly as defined above, wherein the courses pass distally along the flexible airway, such that the dimensions of the at least two sections detached from the airway tube are minimized.

It is another object of the present invention to provide the artificial airway assembly as defined above, wherein at least one of the courses is disposed along the main longitudinal axis.

It is another object of the present invention to provide the artificial airway assembly as defined above, wherein the at least two courses are disposed independently about the circumference of the flexible airway.

It is another object of the present invention to provide the artificial airway assembly as defined above, wherein at least one of the courses is disposed spirally about the circumference of the flexible airway.

It is another object of the present invention to provide the artificial airway assembly as defined above, wherein the courses are homogeneously disposed along the circumference of the flexible airway.

It is another object of the present invention to provide the artificial airway assembly as defined above, wherein the courses are characterized by having two edges overlapping each other.

It is another object of the present invention to provide the artificial airway assembly as defined above, wherein the courses are heterogeneously disposed along the circumference of the flexible airway.

It is another object of the present invention to provide the artificial airway assembly as defined above, wherein the distance between each pair of the plurality of courses is equal.

It is another object of the present invention to provide the artificial airway assembly as defined above, wherein the distance between each pair of the courses is optimized, such that the dimensions of the sections detached from the airway tube are minimized.

It is another object of the present invention to provide the artificial airway assembly as defined above, wherein the distance is calculated according to the artificial airway assembly minimization function F_{Min}(x); the F_{Min}(x) is a function of the number of the courses. n; the circumference of the flexible airway tube, C; volume of the flexible airway tube, V; the distance between each pair of the courses. D, or any combination thereof; represented by F_{Min}(n, C, V, D).

It is another object of the present invention to provide the artificial airway assembly as defined above, wherein the mask comprising at least two courses having predetermined shape, such that at least two separate sections of the airway tube are detachable from the same while maintaining the working tool in the operating position.

It is another object of the present invention to provide the artificial airway assembly as defined above, wherein the at least two courses are complementary to and conform to each other.

It is another object of the present invention to provide the artificial airway assembly as defined above, wherein the courses are locally weakened and tearable courses.

It is another object of the present invention to provide the artificial airway assembly as defined above, wherein the courses are provided so as to minimize the dimensions of the at least one section detached from the mask.

It is another object of the present invention to provide the artificial airway assembly as defined above, wherein the at least one of the courses is disposed independently about the circumference of the mask.

It is another object of the present invention to provide the artificial airway assembly as defined above, wherein the courses are disposed spirally about the circumference of the mask.

It is another object of the present invention to provide the artificial airway assembly as defined above, wherein the courses are characterized by having two edges overlapping each other.

It is another object of the present invention to provide the artificial airway assembly as defined above, wherein the mask is fixedly secured to the operating position of the flexible airway tube.

It is another object of the present invention to provide the artificial airway assembly as defined above, wherein the flexible airway tube is branched.

It is another object of the present invention to provide the artificial airway assembly as defined above, wherein the flexible airway tube is adapted for guiding the working tool to an operating position to view vocal cords and trachea of the patient.

It is another object of the present invention to provide the artificial airway assembly as defined above, further comprising at least one inflatable balloon disposed about, and in physical contact with, one side of the flexible airway, whereby upon inflation of the inflatable balloon while the artificial airway assembly is in place, at least part of the flexible airway in a predetermined direction.

It is another object of the present invention to provide the artificial airway assembly as defined above, wherein the predetermined direction is in the direction of the trachea of the patient.

It is another object of the present invention to provide the artificial airway assembly as defined above, wherein the inflatable balloon is disposed externally to the flexible airway.

It is another object of the present invention to provide the artificial airway assembly as defined above, wherein the inflatable balloon is disposed within the flexible airway.

It is another object of the present invention to provide the artificial airway assembly as defined above, wherein the inflatable balloon is integrated within the flexible airway.

It is another object of the present invention to provide the artificial airway assembly as defined above, wherein the mask is fixedly secured to the distal end of the flexible airway tube, the mask having conforming means for conforming to a space immediately adjacent to the larynx of the patient to properly position the working tool.

It is another object of the present invention to provide the artificial airway assembly as defined above, wherein the predetermined shape of the courses is selected from a group consisting of Zig-Zag shape, waved, sinus, triangular waved shaped, square waved shaped, continuous, discontinued any combination thereof.

It is another object of the present invention to provide an artificial airway assembly for ventilating a patient, the artificial airway assembly (10) comprising: a flexible airway tube having a proximal end and a distal end interconnected by a main longitudinal axis, adapted to guide a working tool to an operating position; at least one inflatable balloon disposed on one side of and in physical contact with the flexible airway tube; and, a mask coupled to the distal end of the flexible airway tube; wherein inflation of the inflatable balloon while the artificial airway assembly is in place, is adapted to direct at least part of the flexible airway to a predetermined location.

It is another object of the present invention to provide the artificial airway assembly as defined above, wherein the predetermined location is the trachea of the patient.

It is another object of the present invention to provide the artificial airway assembly as defined above, wherein the inflatable balloon is disposed externally to the flexible airway.

It is another object of the present invention to provide the artificial airway assembly as defined above, wherein the inflatable balloon is disposed within the flexible airway.

It is another object of the present invention to provide the artificial airway assembly as defined above, wherein the flexible airway tube comprising at least two courses having predetermined shape, such that at least two separate sections of the airway tube are detachable from the same while maintaining the working tool in the operating position.

It is another object of the present invention to provide the artificial airway assembly as defined above, wherein the at least two courses are complementary to and conform to each other.

It is another object of the present invention to provide the artificial airway assembly as defined above, wherein the courses are locally weakened and tearable courses.

It is another object of the present invention to provide the artificial airway assembly as defined above, wherein the courses pass distally along the flexible airway, such that the dimensions of the at least two sections detached from the airway tube are minimized.

It is another object of the present invention to provide the artificial airway assembly as defined above, wherein at least one of the courses is disposed along the main longitudinal axis.

It is another object of the present invention to provide the artificial airway assembly as defined above, wherein the at least two courses are disposed independently about the circumference of the flexible airway.

It is another object of the present invention to provide the artificial airway assembly as defined above, wherein the at least one of the courses is disposed spirally about the circumference of the flexible airway.

It is another object of the present invention to provide the artificial airway assembly as defined above, wherein the plurality of courses is homogeneously disposed along the circumference of the flexible airway.

It is another object of the present invention to provide the artificial airway assembly as defined above, wherein the plurality of courses is heterogeneously disposed along the circumference of the flexible airway.

It is another object of the present invention to provide the artificial airway assembly as defined above, wherein the courses are characterized by having two edges overlapping each other.

It is another object of the present invention to provide the artificial airway assembly as defined above, wherein the distance between each pair of the plurality of courses is equal.

It is another object of the present invention to provide the artificial airway assembly as defined above, wherein the distance between each pair of the courses is optimized, such that the dimensions of the sections detached from the airway tube are minimized.

It is another object of the present invention to provide the artificial airway assembly as defined above, wherein the distance is calculated according to the artificial airway assembly minimization function F_{Min}(x); the F_{Min}(x) is a function of the number of the courses. n; the circumference of the flexible airway tube, C; volume of the flexible airway tube, V; the distance between each pair of the courses. D, or any combination thereof; represented by F_{Min}(n, C, V, D).

It is another object of the present invention to provide the artificial airway assembly as defined above, wherein the mask comprising at least two courses having predetermined shape, such that at least two separate sections of the airway tube are detachable from the same while maintaining the working tool in the operating position.

It is another object of the present invention to provide the artificial airway assembly as defined above, wherein the at least two courses are complementary to and conform to each other.

It is another object of the present invention to provide the artificial airway assembly as defined above, wherein the courses are locally weakened and tearable courses.

It is another object of the present invention to provide the artificial airway assembly as defined above, wherein the courses are provided so as to minimize the dimensions of the at least one section detached from the mask.

It is another object of the present invention to provide the artificial airway assembly as defined above, wherein the at least one of the courses is disposed independently about the circumference of the mask.

It is another object of the present invention to provide the artificial airway assembly as defined above, wherein the at least one of the courses is disposed spirally about the circumference of the flexible airway.

It is another object of the present invention to provide the artificial airway assembly as defined above, wherein the courses are characterized by having two edges overlapping each other.

It is another object of the present invention to provide the artificial airway assembly as defined above, wherein the mask is fixedly secured to the operating position of the flexible airway tube.

It is another object of the present invention to provide the artificial airway assembly as defined above, wherein the flexible airway tube is branched.

It is another object of the present invention to provide the artificial airway assembly as defined above, wherein the flexible airway tube is adapted for guiding the working tool to an operating position to view vocal cords and trachea of the patient.

It is another object of the present invention to provide the artificial airway assembly as defined above, wherein mask coupled fixedly secured to the distal end of the flexible airway tube, the mask having conforming means for conforming to a space immediately adjacent to the larynx of the patient to properly position the working tool.

It is another object of the present invention to provide the artificial airway assembly as defined above, wherein the predetermined shape of the courses is selected from a group consisting of Zig-Zag shape, waved, sinus, triangular waved shaped, square waved shaped, continuous, discontinuous or any combination thereof.

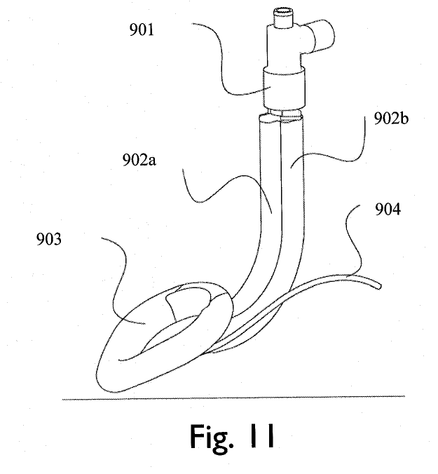

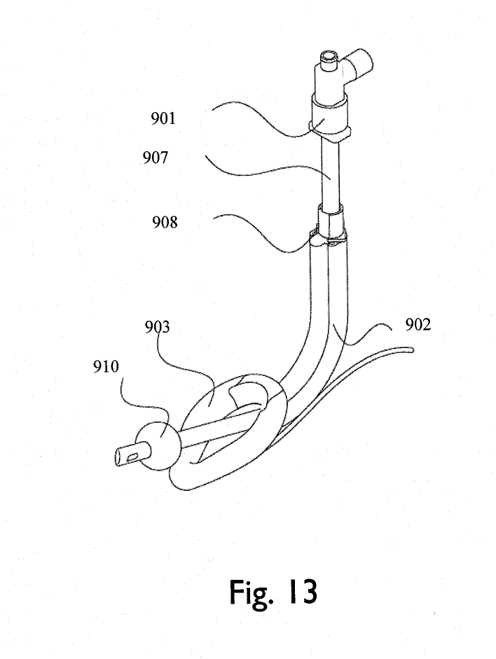

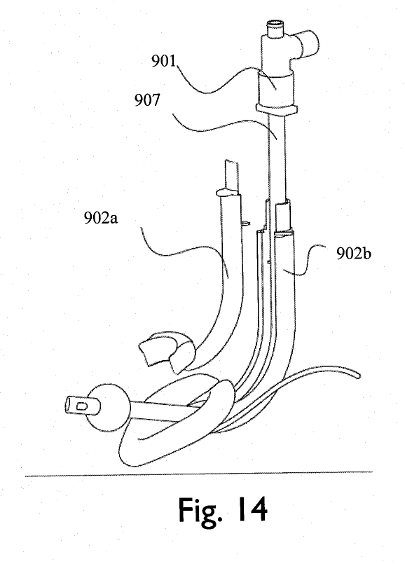

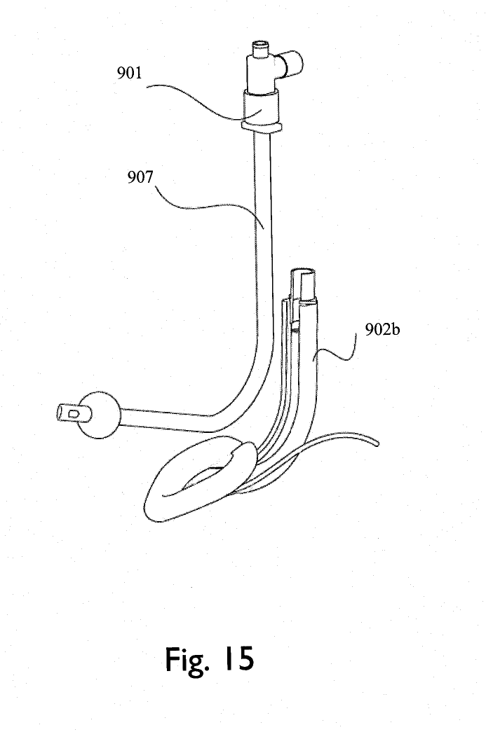

Reference is made to FIGS. 11-15 which illustrate another embodiment of the present invention which illustrate a drawer-like configuration of the mask tube.

FIGS. 11-15 schematically present an artificial airway assembly for ventilating a patient, the artificial airway assembly comprising: a flexible airway tube 902 having a proximal end and a distal end, the proximal end comprising a pass-through means 901a for allowing a working tool to pass there through and for guiding the working tool to an operating position to view vocal cords and trachea of the patient; at least one airway-directing mechanism disposed on one side of and in physical contact with the flexible airway tube; and, a mask 903 coupled to the distal end of the flexible airway tube.

The flexible airway tube comprises a dorsal 902a section and a venal section 902b; the flexible airway tube 902 has a drawer-like configuration 902a-b for detachment of at least a section 902a of the airway tube while maintaining the working tool in the operating position; further wherein the activation of the airway-directing mechanism while the artificial airway assembly is in place, is directing at least part of the flexible airway to a predetermined location.

It is still object of the present invention to provide the artificial airway assembly as defined above, wherein the drawer like configuration of the flexible airway tube 902 comprises a drawn section 902a allowing extraction of the laryngeal mask airway tube 902a-b while maintaining the flexible airway tube 907 in the operating position.

It is still object of the present invention to provide the artificial airway assembly as defined above, wherein the drawer like configuration of the flexible airway tube is characterized by having two connecting sections overlapping each other.

It is still object of the present invention to provide the artificial airway assembly as defined above, wherein the detachment section of the airway tube is the ventral section 902a.

It is still object of the present invention to provide the artificial airway assembly as defined above, wherein the detachment of at least a section of the airway tube is preformed in manner selected from the group consisting of a sliding mechanism, a lego-like mechanism, pull out mechanism, quick release mechanism, or any disengaging mechanism.

The present invention further provides the connecting manner of the dorsal 902b and ventral 902a section of the artificial airway assembly selected from a group consisting of a sliding mechanism, a lego-like mechanism, quick fit mechanism, or any engaging mechanism allowing a firmly fit connection.

The artificial airway assembly defined above, is possibly provided with a pneumatic airway-directing mechanism which comprises at least one inflatable balloon useful to direct at least part of the flexible airway to a predetermined location. Additionally or alternatively, the artificial airway assembly defined above, is possibly provided with a mechanical airway-directing mechanism which comprises at least one maneuverable effecter useful to direct at least part of the flexible airway to a predetermined location. An artificial airway assembly which comprises an integrated mechanism, namely a pneumatic and mechanical mechanism is possible.

FIG. 11-15 further comprises an inflatable balloon 910 disposed about one side of, and in physical contact with, the flexible airway tube. When the assembly is inserted, inflatable balloon is in its deflated state. In the preferred embodiment of the invention shown in FIG. 11-15, The assembly further comprises inflation tube in fluid communication with balloon 905. Any other means for inflating the balloon known in the art may be used.

The assembly further comprises an inflation tube 904 in fluid communication with the mask of the present invention.

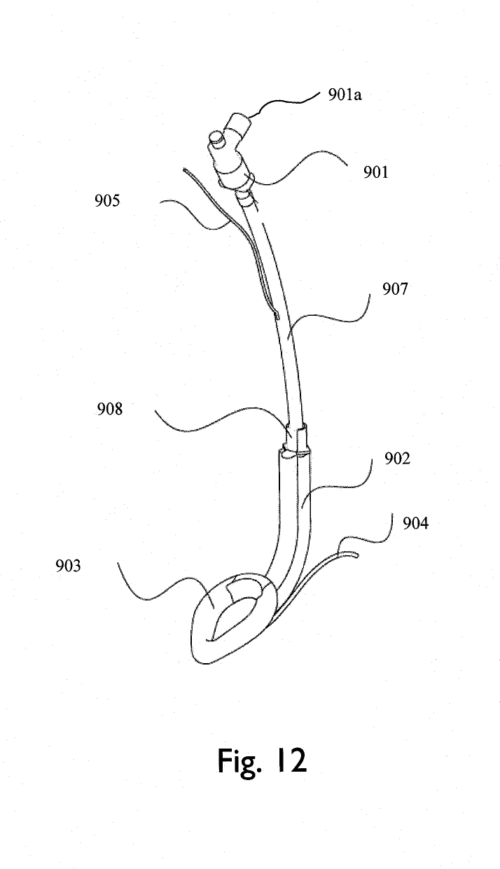

The tracheal airway tube presented in FIG. 12 further comprise a L shape fitting part 901 having a bushing part for adjusting of the airway tube within the laryngeal mask tube when positioned in the trachea. The bushing part is further adjustable by a thread. The L shape fitting part comprises a brunch 901a for guiding a working tool into the flexible airway tube and allows for ventilation to continue without interruption through the flexible airway tube. When the tracheal airway tube is positioned within the trachea the valve 901 is adjusted to a thread sleeve 908 located in the proximal portion of the flexible airway tube.

It is another object of the present invention to provide a method for intubating a patient using an artificial airway assembly, the method comprising a step of a. obtaining a flexible airway tube having a proximal end and a distal end interconnected by a main longitudinal axis; adapted to guide a working tool to an operating position; the flexible airway tube comprising at least two courses. having predetermined shape, along which at least two sections of the airway tube are detached while maintaining the working tool in the operating position; b. fixedly securing a mask to the distal end of the flexible airway tube; c. inserting the artificial airway assembly into the mouth of a patient; d. positioning the mask adjacent to the larynx; e. moving the working tool through the flexible airway tube at least to the distal end of the artificial airway assembly and the mask; f. at least partially tearing the flexible airway tube along the at least two courses. thereby detaching at least two sections of the airway tube while maintaining the working tool in the operating position; and, g. removing the artificial airway assembly from the mouth of the patient.

It is another object of the present invention to provide the method as defined above, additionally comprising a step of initiating ventilation of the patient via the proximal end.

It is another object of the present invention to provide the method as defined above, in which the step of moving the working tool through the flexible airway tube further comprises a step of moving the working tool beyond the mask and into the trachea of the patient.

It is another object of the present invention to provide the method as defined above, wherein the step of removing the artificial airway assembly from the working tool further comprises steps of: tearing the flexible airway tube along each of the courses. and, removing the artificial airway assembly in a piecewise fashion as each piece is detached.

It is another object of the present invention to provide the method as defined above, wherein the at least two courses are complementary to and conform to each other.

It is another object of the present invention to provide the method as defined above, wherein the courses are locally weakened and tearable courses.

It is another object of the present invention to provide the method as defined above, wherein the artificial airway assembly further comprises an inflatable balloon disposed about one side of the flexible airway tube, the method further comprising a step of at least partially inflating the balloon whereby the flexible airway tube is directed toward the trachea of the patient.

It is another object of the present invention to provide the method as defined above, further including a step of deflating the inflatable balloon.

It is another object of the present invention to provide the method as defined above, additionally comprising a step of disposing the courses along the flexible airway, such that the dimensions of the two sections detached from the airway tube are minimized.

It is another object of the present invention to provide the method as defined above, additionally comprising a step of disposing at least one of the courses along the main longitudinal axis.

It is another object of the present invention to provide the method as defined above, additionally comprising a step of disposing the courses independently about the circumference of the flexible airway.

It is another object of the present invention to provide the method as defined above, additionally comprising a step of disposing the courses comprises are disposed spirally about the circumference of the flexible airway.

It is another object of the present invention to provide the method as defined above, additionally comprising a step of homogeneously disposing the courses along the circumference of the flexible airway.

It is another object of the present invention to provide the method as defined above, additionally comprising a step of heterogeneously disposing the plurality of courses along the circumference of the flexible airway.

It is another object of the present invention to provide the method as defined above, additionally comprising a step of providing the courses by having two edges overlapping each other.

It is another object of the present invention to provide the method as defined above, wherein the distance between each pair of the courses is equal.

It is another object of the present invention to provide the method as defined above, wherein the distance between each pair of the courses is optimized, such that the dimensions of the sections detached from the airway tube are minimized.

It is another object of the present invention to provide the method as defined above, wherein the distance is calculated according to the artificial airway assembly minimization function F_{Min}(x); the F_{Min}(x) is a function of the number of the courses. n; the circumference of the flexible airway tube, C; volume of the flexible airway tube, V; the distance between each pair of the courses. D, or any combination thereof; represented by F_{Min}(n, C, V, D).

It is another object of the present invention to provide the method as defined above, additionally comprising a step of providing the mask with at least two courses having predetermined shape, such that at least two separate sections of the airway tube are detachable from the same while maintaining the working tool in the operating position.

It is another object of the present invention to provide the method as defined above, wherein the at least two courses are complementary to and conform to each other.

It is another object of the present invention to provide the method as defined above, wherein the courses are locally weakened and tearable courses.

It is another object of the present invention to provide the method as defined above, additionally comprising a step of providing the courses, so as to minimize the dimensions of the at least one section detached from the mask.

It is another object of the present invention to provide the method as defined above, wherein at least one of the courses are disposed independently about the circumference of the mask.

It is another object of the present invention to provide the method as defined above, additionally comprising a step of fixedly securing the mask to the operating position of the flexible airway tube.

It is another object of the present invention to provide the method as defined above, wherein the flexible airway tube is branched.

It is another object of the present invention to provide the method as defined above, wherein the flexible airway tube is adapted for guiding the working tool to an operating position to view vocal cords and trachea of the patient.

It is another object of the present invention to provide the method as defined above, additionally comprising a step of providing at least one inflatable balloon disposed about, and in physical contact with, one side of the flexible airway.

It is another object of the present invention to provide the method as defined above, additionally comprising a step of, upon inflation of the inflatable balloon while the artificial airway assembly is in place, directing at least part of the flexible airway in the direction of the trachea of the patient.

It is another object of the present invention to provide the method as defined above, additionally comprising a step of disposing the inflatable balloon externally to the flexible airway.

It is another object of the present invention to provide the method as defined above, additionally comprising a step of disposing the inflatable balloon within the flexible airway.

It is another object of the present invention to provide the method as defined above, additionally comprising a step of integrating the inflatable balloon within the flexible airway.

It is still object of the present invention to provide the method as defined above, additionally comprising a step of providing the mask with conforming means for conforming to a space immediately adjacent to the larynx of the patient to properly position the working tool.

It is lastly an object of the present invention to provide the method as defined above, wherein the predetermined shape of the courses is selected from a group consisting of Zig-Zag shape, waved, sinus, triangular waved shaped, square waved shaped, continuous, and discontinuous or any combination thereof.

1-107. (canceled)

108. An artificial airway assembly for ventilating a patient, said artificial airway assembly comprising:
a flexible airway tube having a proximal end and a distal end, said proximal end comprising a pass-through means for allowing a working tool to pass there through and for guiding the working tool to an operating position to view vocal cords and trachea of the patient, said flexible airway tube comprising at least two locally weakened and tearable courses adapted to permit detachment of at least a section of said airway tube while maintaining said working tool in said operating position; and
a mask fixedly secured to said distal end of said flexible airway tube, said mask having conforming means for conforming to a space immediately adjacent to the larynx of the patient to properly position said working tool.

109. The artificial airway assembly according to claim 108, wherein at least one of the following is being held true (a) said at least two courses are complementary to and conform to each other; (b) said courses are locally weakened and tearable courses; (c) said courses pass distally along said flexible airway; (d) at least one of said courses is disposed along said main longitudinal axis; (e) said at least two courses are disposed independently about the circumference of said flexible airway; (f) at least one of said courses is disposed spirally about the circumference of said flexible airway; (g) said courses are homogeneously disposed along the circumference of said flexible airway; (h) said courses are characterized by having two edges overlapping each other; (i) said courses are heterogeneously disposed along the circumference of said flexible airway; (j) the distance between each pair of said plurality of courses is equal; and any combination thereof.

110. The artificial airway assembly according to claim 108, wherein at least one of the following is being held true (a) said mask comprising at least two courses having predetermined shape, such that at least two separate sections of said airway tube are detachable from the same while maintaining said working tool in said operating position; (b) said at least two courses are complementary to and conform to each other; (c) said courses are locally weakened and tearable courses; (d) said courses are provided so as to minimize the dimensions of said at least one section detached from said mask; (e) said at least one of said courses is disposed independently about the circumference of said mask; (f) said courses are disposed spirally about the circumference of said mask; (g) said courses are characterized by having two edges overlapping each other; and any combination thereof.

111. The artificial airway assembly according to claim 108, wherein at least one of the following is being held true (a) said mask is fixedly secured to said operating position of said flexible airway tube; (b) said flexible airway tube is branched; (c) said flexible airway tube is adapted for guiding the working tool to an operating position to view vocal cords and trachea of said patient; (c) said mask is fixedly secured to said distal end of said flexible airway tube, said mask having conforming means for conforming to a space immediately adjacent to the larynx of said patient to properly position said working tool; (d) said predetermined shape of said courses is selected from a group consisting of Zig-Zag shape, waved, sinus, triangular waved shaped, square waved shaped, continuous, discontinuous or any combination thereof; (e) said artificial airway assembly additionally comprising a maneuvering mechanism adapted to reorient the distal end of said flexible airway tube; and any combination thereof.

112. The artificial airway assembly according to claim 108, further comprising at least one inflatable balloon disposed about, and in physical contact with, one side of said flexible airway, whereby upon inflation of said inflatable balloon while said artificial airway assembly is in place, at least part of said flexible airway is provided in a predetermined direction.

113. The artificial airway assembly according to claim 112, wherein at least one of the following is being held true (a) said predetermined direction is in the direction of the trachea of said patient; (b) said inflatable balloon is disposed externally to said flexible airway; (c) said inflatable balloon is disposed within said flexible airway; (d) said inflatable balloon is integrated within said flexible airway; and any combination thereof.

114. An artificial airway assembly for ventilating a patient, said artificial airway assembly comprising:
a flexible airway tube having a proximal end and a distal end, said proximal end comprising a pass-through means for allowing a working tool to pass there through and for guiding the working tool to an operating position to view vocal cords and trachea of the patient;
at least one airway-directing mechanism disposed on one side of and in physical contact with said flexible airway tube; and,
a mask coupled to said distal end of said flexible airway tube;
wherein said flexible airway tube comprises a dorsal section and a ventral section; said flexible airway tube has a drawer-like configuration for detachment of at least a section of said airway tube while maintaining said working tool in said operating position; further wherein said activation of said airway-directing mechanism while said artificial airway assembly is in place, is directing at least part of said flexible airway to a predetermined location.

115. The artificial airway assembly according to claim 114, wherein at least one of the following is being held true (a) said drawer like configuration of said flexible airway tube comprises a drawn section allowing extraction of said mask tube while maintaining said flexible airway tube in said operating position; (b) said drawer like configuration of said flexible airway tube is characterized by having two connecting sections overlapping each other; (c) said detachment section of said airway tube is the ventral section; (d) said detachment of at least a section of said airway tube is preformed in manner selected from the group consisting of: a sliding mechanism, a lego-like mechanism, pull out mechanism, quick release mechanism, or any disengaging mechanism; (e) said airway-directing mechanism is selected from a group consisting of pneumatic, mechanic and pneumatic-mechanic integrated mechanism; (f) said pneumatic airway-directing mechanism comprising at least one inflatable balloon useful to direct at least part of said flexible airway to a predetermined location; (g) said mechanic airway-directing mechanism comprising at least one maneuverable effecter useful to direct at least part of said flexible airway to a predetermined location; said predetermined location is said trachea of said patient; (h) said flexible airway tube comprising at least two courses having predetermined shape, such that at least two separate sections of said airway tube are detachable from the same while maintaining said working tool in said operating position; (i) said at least two courses are complementary to and conform to each other; (j) said courses are locally weakened and tearable courses; (h) at least one of said courses is disposed along said main longitudinal axis; (i) said at least two courses are disposed independently about the circumference of said flexible airway; (j) said at least one of said courses is disposed spirally about the circumference of said flexible airway; (k) said plurality of courses are homogeneously disposed along the circumference of said flexible airway; and any combination thereof.

116. The artificial airway assembly according to claim 114, wherein at least one of the following is being held true (a) said plurality of courses are heterogeneously disposed along the circumference of said flexible airway; (b) said courses are characterized by having two edges overlapping each other; (c) the distance between each pair of said plurality of courses is equal; (d) said mask comprises at least two courses having predetermined shape, such that at least two separate sections of said airway tube are detachable from the same while maintaining said working tool in said operating position; and any combination thereof.

117. The artificial airway assembly according to claim 114, wherein at least one of the following is being held true (a) said mask is fixedly secured to said operating position of said flexible airway tube; (b) said flexible airway tube is branched; (c) said flexible airway tube is adapted for guiding the working tool to an operating position to view vocal cords and trachea of said patient; (d) said mask coupled fixedly secured to said distal end of said flexible airway tube, said mask having conforming means for conforming to a space immediately adjacent to the larynx of the patient to properly position said working tool; (e) said predetermined shape of said courses is selected from a group consisting of Zig-Zag shape, waved, sinus, triangular waved shaped, square waved shaped, continuous, discontinuous or any combination thereof; (f) additionally comprising a maneuvering mechanism adapted to reorient the distal end of said flexible airway tube; and any combination thereof.

118. A method of intubating a patient using an artificial airway assembly, said method comprising:
a. obtaining a flexible airway tube having a proximal end and a distal end, said proximal end comprising a pass-through means for allowing a working tool to pass there through and for guiding the working tool to an operating position to view vocal cords and trachea of the patient, said flexible airway tube comprising at least two locally weakened and tearable courses adapted to permit detachment of at least a section of said airway tube while maintaining said working tool in said operating position;
b. fixedly securing a mask to said distal end of said flexible airway tube;
c. inserting the artificial airway assembly into the mouth of a patient;
d. positioning said mask adjacent to the larynx;
e. moving said working tool through said flexible airway tube at least to the distal end of said artificial airway assembly and said mask;
f. at least partially tearing said flexible airway tube along said at least two courses. thereby detaching at least two sections of said airway tube while maintaining said working tool in said operating position; and
g. removing said artificial airway assembly from the mouth of said patient.

119. The method according to claim 118, additionally comprising at least one step selected from a group consisting of (a) initiating ventilation of the patient via the proximal end; (b) disposing at least one of said courses along said main longitudinal axis; (c) disposing said courses independently about the circumference of said flexible airway; (d) disposing said courses spirally about the circumference of said flexible airway; (e) homogeneously disposing said courses along the circumference of said flexible airway; (f) heterogeneously disposing said plurality of courses along the circumference of said flexible airway; and any combination thereof.

120. The method according to claim 118, in which said step of moving said working tool through said flexible airway tube further comprises a step of moving said working tool beyond said mask and into the trachea of said patient.

121. The method according to claim 118, wherein said step of removing said artificial airway assembly from said working tool further comprises steps of:
tearing said flexible airway tube along each of said courses; and,
removing said artificial airway assembly in a piecewise fashion as each piece is detached.

122. The method according to claim 118, wherein at least one of the following is being held true (a) said at least two courses are complementary to and conform to each other; (b) said courses are locally weakened and tearable courses; (c) said artificial airway assembly further comprises an airway-directing mechanism disposed about one side of said flexible airway tube, said method further comprising a step of at least partially maneuvering said airway-directing mechanism whereby said flexible airway tube is directed toward the trachea of said patient.

123. The method according to claim 118, additionally comprising at least oen step selected from a group consisting of (a) providing said courses by having two edges overlapping each other; (b) providing said mask with at least two courses having predetermined shape, such that at least two separate sections of said airway tube are detachable from the same while maintaining said working tool in said operating position; (c) providing said courses so as to minimize the dimensions of said at least one section detached from said mask; and any combination thereof.

124. The method according to claim 118, wherein at least one of the following is being held true (a) said at least two courses are complementary to and conform to each other; (b) said courses are locally weakened and tearable courses; (c) at least one of said courses are disposed independently about the circumference of said mask; (d) said flexible airway tube is branched; (e) said flexible airway tube is adapted for guiding the working tool to an operating position to view vocal cords and trachea of said patient; and any combination thereof.

125. The method according to claim 118, additionally comprising at least one step selected from a group consisting of (a) fixedly securing said mask to said operating position of said flexible airway tube; (b) of providing at least one inflatable balloon disposed about, and in physical contact with, one side of said flexible airway; (c) directing at least part of said flexible airway in the direction of the trachea of said patient; (d) disposing said inflatable balloon externally to said flexible airway; (e) disposing said inflatable balloon within said flexible airway; and any combination thereof.

126. The method according to claim 118, additionally comprising at least one step selected from a group consisting of (a) integrating said inflatable balloon within said flexible airway; (b) providing said mask with conforming means for conforming to a space immediately adjacent to the larynx of the patient to properly position said working tool; (c) maneuvering and reorienting the distal end of said flexible airway tube; and any combination thereof.

127. The method according to claim 118, wherein said predetermined shape of said locally weakened and tearable courses is selected from a group consisting of Zig-Zag shape, waved, sinus, triangular waved shaped, square waved shaped, continuous, discontinuous or any combination thereof.
